(19) United States
(12) Patent Application Publication
(10) Pub. No.: US 2009/0221885 A1
(43) Pub. Date: Sep. 3, 2009

(54) Optical Window Assembly for Implantable Medical Device

(75) Inventors: Peter Hall, Andover, MN (US); Daniel J. Cooke, Roseville, MN (US); Michael John Kane, Clonmel (IE)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: Cardiac Pacemakers, Inc., St. Paul, MN (US)

(21) Appl. No.: 12/391,761

(22) Filed: Feb. 24, 2009

Related U.S. Application Data
(60) Provisional application No. 61/031,126, filed on Feb. 25, 2008.

(51) Int. Cl. A61B 5/1473; B23K 31/02

(52) U.S. Cl. 600/309; 228/175

(57) ABSTRACT

Embodiments of the invention are related to optical window assemblies for implantable medical devices, amongst other things. In an embodiment, the invention includes an optical window assembly for a medical device. The assembly can include a ferrule defining an aperture and a spacer ring disposed within the aperture. The assembly can also include an optical window coupled to the metal ferrule and the spacer ring. In an embodiment, the invention includes an implantable medical device including a housing and an optical window assembly coupled to the housing. In an embodiment, the invention can include a method of manufacturing a medical device. The method can include brazing a spacer ring to a metal ferrule, coupling an optical window to the spacer ring and the metal ferrule with a bonding glass material, depositing a chemical sensing element over the optical window, and coupling a porous cover layer to the spacer ring and the ferrule with an adhesive. Other embodiments are also included herein.

### RELATED APPLICATIONS

This application claims priority to provisional U.S. patent application 61/031,126, filed Feb. 25, 2008, the contents of which are herein incorporated by reference.

### TECHNICAL FIELD

This disclosure relates generally to optical window assemblies and, more particularly, to optical window assemblies for implantable medical devices, amongst other things.

### BACKGROUND OF THE INVENTION

Certain physiological analytes are relevant to the diagnosis and treatment of medical problems. As one example, potassium ion concentrations can affect a patient's cardiac rhythm. Therefore, medical professionals frequently evaluate physiological potassium ion concentration when diagnosing cardiac rhythm problems. However, measuring physiological concentrations of analytes, such as potassium, generally requires drawing blood from the patient followed by analysis with in vitro techniques. Blood draws generally require the patient to physically visit a medical facility, such as a hospital or clinic. As a result, despite their medical significance, physiological analyte concentrations are frequently measured less often than desired due to patient discomfort and inconvenience.

One solution to these issues is to use an implanted sensor to measure physiological concentrations of analytes of interest. As such, significant efforts have been directed at the development of suitable implantable sensors. However, chronically implantable sensors present challenging design issues. In particular, chronically implantable sensors designed to use optical techniques to sense analytes present challenging design issues.

### SUMMARY OF THE INVENTION

Embodiments of the invention are related to optical window assemblies for implantable medical devices, amongst other things. In an embodiment, the invention includes an optical window assembly for a medical device. The assembly can include a ferrule defining an aperture and a spacer ring disposed within the aperture. The spacer ring can be coupled to the ferrule with a brazing material. The assembly can also include an optical window coupled to the metal ferrule and the spacer ring with a bonding glass material. The optical window can have a coefficient of thermal expansion within approximately 3.0×10⁻⁶ inch per inch/° C. of the coefficient of thermal expansion of the spacer ring.

In an embodiment, the invention includes an implantable medical device including a hermetically sealed housing defining an interior volume and an optical window assembly coupled to the housing. The optical window assembly can include a ferrule defining an aperture and a spacer ring disposed within the aperture. The spacer ring can be coupled to the ferrule with a brazing material. The assembly can also include an optical window coupled to the metal ferrule and the spacer ring with a bonding glass material. The optical window can have a coefficient of thermal expansion within approximately 3.0×10⁻⁶ inch per inch/° C. of the coefficient of thermal expansion of the spacer ring.

In an embodiment, the invention can include a method of manufacturing a medical device. The method can include brazing a spacer ring to a metal ferrule, coupling an optical window to the spacer ring and the metal ferrule with a bonding glass material, depositing a chemical sensing element over the optical window, and coupling a porous cover layer to the spacer ring and the ferrule with an adhesive.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is a schematic view of an implantable medical device in accordance with at least one embodiment.

FIG. 2 is an exploded view of an optical window assembly in accordance with at least one embodiment.

FIG. 3 is a top schematic view of an optical window assembly in accordance with at least one embodiment.

FIG. 4 is a cross sectional schematic view of an optical window assembly as taken along line 4-4 of FIG. 3.

FIG. 5 is a cross sectional schematic view of an optical window assembly in accordance with at least one embodiment.

FIG. 6 is a cross sectional schematic view of an optical window assembly in accordance with at least one embodiment.

FIG. 7 is a cross sectional schematic view of an optical window assembly in accordance with at least one embodiment.

FIG. 8 is a cross sectional schematic view of an optical window assembly in accordance with at least one embodiment.

FIG. 9 is a cross sectional schematic view of an optical window assembly in accordance with at least one embodiment.

FIG. 10 is a top view of an optical window assembly in accordance with at least one embodiment.

FIG. 11 is a top view of an optical window assembly in accordance with at least one embodiment.

FIG. 12 is an exploded view of an optical window assembly in accordance with at least one embodiment.

FIG. 13 is a flow diagram of an exemplary method in accordance with at least one embodiment.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Information regarding the concentration of physiological analytes can be important to the diagnosis and treatment of many medical problems. For example, knowledge of potassium ion concentrations can be important to the correct diagnosis of cardiac arrhythmias. Likewise, the concentrations of other physiological ions, such as sodium and calcium, can also be important in the diagnosis and treatment of cardiac arrhythmias.

Knowledge of the concentration of physiological analytes can also be useful in the context of monitoring drug therapy, monitoring renal function, titrating drugs (such as heart failure medications), monitoring heart failure, and observing primary electrolyte imbalance subsequent to dietary intake or renal excretion variations, amongst other uses.

The use of implantable sensors to gather data regarding physiological analyte concentrations can be particularly valuable because the data can be gathered as often as desired without inconveniencing the patient. In addition, implantable sensors offer the advantage of being able to gather data in real time.

However, implantable sensor systems, and particularly implantable optical sensor systems, pose various challenging design issues. Such issues include protecting sensitive electronic components within a hermetically sealed environment while also preventing potentially non-biocompatible materials from contacting tissues of the subject.

Various embodiments described herein can address these design issues. In an embodiment, the invention includes an optical window assembly for a medical device. The optical window assembly can include a ferrule defining an aperture. The optical window assembly can also include a spacer ring disposed within the aperture and coupled to the ferrule. The spacer ring can be made of a high strength material so that pressure from thermal expansion and contraction of the ferrule will be absorbed by the spacer ring without resulting in structural failure. The optical window assembly can also include an optical window coupled to the ferrule and the spacer ring. The optical window, generally made from a material with less strength than the spacer ring and therefore more susceptible to structural failure, can have a coefficient of thermal expansion that closely matches that of the spacer ring. In this manner, thermal expansion and contraction of the spacer ring can be roughly equivalent to that of the optical window and therefore expansion and contraction of the spacer ring due to fluctuations in temperature will not result in significant forces being applied to the optical window.

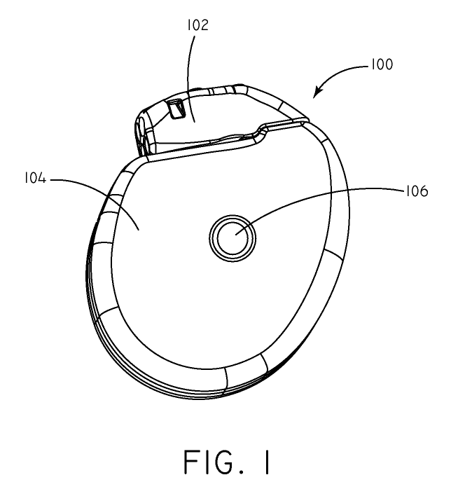

Various embodiments will now be described in greater detail. Referring now to FIG. 1, a schematic view is shown of an implantable medical device in accordance with at least one embodiment. The implantable medical device 100 includes a housing 104 (or can) and a header assembly 102 coupled to the housing 104. The housing 104 can include various materials such as metals, polymers, ceramics, and the like. In a particular embodiment, the housing 104 is formed of titanium. The header assembly 102 serves to provide fixation of the proximal end of one or more leads (not shown) and couples the leads, optically or electrically, to components within the housing 104. The header assembly 102 can be formed of various materials including metals, polymers, ceramics, and the like.

The implantable medical device 100 can be a pacemaker, cardioverter-defibrillator, monitoring device, or the like. By way of example, some aspects of exemplary devices are described in U.S. Pat. No. 6,928,325, issued Aug. 9, 2005, the content of which is herein incorporated by reference. The implantable medical device 100 also includes an optical window assembly 106.

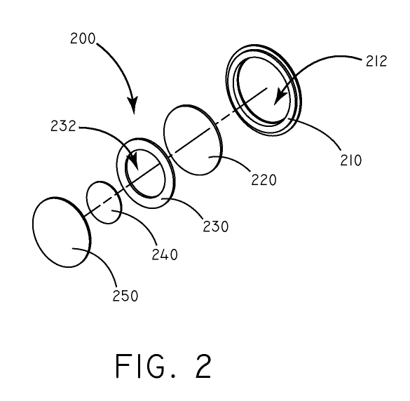

FIG. 2 is an exploded view of an optical window assembly 200 for implantable sensors in accordance with at least one embodiment. The optical window assembly 200 can include a ferrule 210. The ferrule 210 can be constructed from various materials. By way of example, in some embodiments, the ferrule 210 can include a metal. Exemplary metals can include titanium, platinum, chromium, various alloys, and the like. In some embodiments the ferrule 210 is constructed from a substantially biocompatible material. In some embodiments, the ferrule 210 comprises the same material as the housing of the implantable medical device that the ferrule 210 is configured to engage. However, in other embodiments the ferrule 210 can comprise a different material than the housing of the implantable medical device. The ferrule 210 can be made by machining, coining, stamping, deep drawing, or through another technique. The ferrule defines an aperture 212.

The optical window assembly 200 can also include a porous cover 220. In some embodiments, the porous cover 220 can be at least partially disposed in the aperture 212 defined by the ferrule 210. In other embodiments, the porous cover 220 can be disposed outside of the aperture 212. The porous cover 220 can be a porous substrate that allows ingress of analytes of interest. The porous cover 220 can be constructed of a substantially biocompatible material. The porous cover 220 can be made from a ceramic, a metal, a polymer, or combinations thereof. In some embodiments, the porous cover 220 can comprise, for example, porous platinum, porous titanium, porous stainless steel, porous alumina, sintered titanium, sintered iridium, sintered gold or the like. In one embodiment, the porous cover 220 comprises platinum-electro-plated porous alumina. The porous cover 220 can also include an etched substrate (such as dense alumina etched with hydrofluoric acid to remove residual glass) or a substrate that has been laser-cut and/or drilled. In some embodiments, the pores in the porous cover 220 have a diameter of less than about 5 microns. In various embodiments, the pores in the porous cover 220 have a diameter of less than about 1 micron. In some embodiments, the pores in the porous cover 220 have a diameter of less than about 0.5 microns.

The porous cover 220 can be configured to protect or seclude various elements included in the assembly described herein. For example, the porous cover 220 can be opaque to particular spectra of light, such as visible light and/or infrared light, which can prevent interference with optical sensing operations.

The optical window assembly 200 can also include a spacer ring 230. The spacer ring can define a cavity 232. The spacer ring 230 can be disposed within the aperture 212 defined by the ferrule 210. In this configuration, the spacer ring 230 can be positioned to absorb an applied load resulting from thermal expansion or contraction of the ferrule 210. The spacer ring 230 can include a high strength material. In some embodiments, the spacer ring 230 comprises a ceramic. By way of example, the spacer ring 230 can include zirconia and/or alumina. In one embodiment the spacer ring 230 is cut from an extruded high-purity, fully dense alumina tube. In some embodiments, the spacer ring 230 can include carbides, glass ceramics, and/or aluminum silicates (such as Mullite).

The optical window assembly 200 can include a chemical sensing element 240. The chemical sensing element 240 can be at least partially disposed within the cavity 232 defined by the spacer ring 230. The chemical sensing element 240 can be configured to detect one or more analytes of interest. In some embodiments, the chemical sensing element 240 is configured to detect analytes relevant to the health of a person or animal. For example, the chemical sensing element 240 can be configured to detect one or more analytes relevant to cardiac health and/or renal health. Specific analytes that the chemical sensing element 240 can be configured to detect can include, but are not limited to one or more of acetic acid (acetate), aconitic acid (aconitate), ammonium, blood urea nitrogen (BUN), B-type natriuretic peptide (BNP), bromate, calcium ion, carbon dioxide, cardiac specific troponin, chloride, choline, citric acid (citrate), cortisol, copper ion, creatinine, creatinine kinase, fluoride, formic acid (formate), glucose, hydronium ion, isocitrate, lactic acid (lactate), lithium ion, magnesium ion, maleic acid (maleate), malonic acid (malonate), myoglobin, nitrate, nitric-oxide, oxalic acid (oxalate), oxygen, phosphate, phthalate, potassium, pyruvic acid (pyruvate), selenite, sodium ion, sulfate, urea, uric acid, zinc ion, hydronium ion, lithium ion, sodium ion, potassium ion, magnesium ion, calcium ion, silver ion, zinc ion, mercury ion, lead ion, carbonate anion, nitrate anion, sulfite anion, iodide anion, norephedrine, ephedrine, amphetamine, procaine, prilocalne, lidocaine, bupivacaine, lignocaine, creatinine, protamine, salicylate, phthalate, maleate, heparin, ammonia, ethanol, and various organic amines.

The chemical sensing element 240 can be configured to change one or more properties upon exposure to one or more particular analytes. As an example, the chemical sensing element 240 can change optical properties.

In some embodiments, an analyte is detected directly. In other embodiments, an analyte is detected indirectly. By way of example, a metabolite of a particular analyte can be detected instead of the particular analyte itself. In other embodiments, an analyte can be chemically converted into another form, such as an ion, in order to make the process of detection easier. By way of example, an enzyme can be used to convert an analyte into another compound which is easier to detect. Aspects of exemplary sensing elements are described in U.S. patent application Ser. No. 11/383,933, the content of which is herein incorporated by reference.

The optical window assembly 200 can include an optical window 250. The optical window 250 can, in some embodiments, be a cut and polished optical window. The optical window 250 can be planar. In some embodiments, the optical window 250 is substantially rigid. However, in other embodiments, the optical window 250 is flexible. The optical window 250 can be configured to allow or disallow particular wavelengths or wavelength ranges of electromagnetic radiation. The optical window 250 can have one or more surfaces coated with an anti-reflective coating in order to reduce reflective loss through the optical window 250. The optical window 250 also can have a coating disposed thereon to filter out particular electromagnetic wavelengths or wavelength ranges. Various materials can be used to form the optical window 250 including crystal, glass, ceramics, polymers, and the like. In some embodiments, the optical window 250 can include, but is not limited to, materials such as sapphire (aluminum oxide), soda lime glass, and borosilicate glass. In some embodiments, the optical window 250 can include polyurethane. In various embodiments, the optical window 250 can be made of a biocompatible material.

It can be desirable to limit the applied forces on the optical window 250 in order to reduce the chances of structural failure of the optical window 250, such as through cracking or fracturing. In some embodiments, the optical window 250 has a coefficient of thermal expansion that is substantially similar to the spacer ring 230. As such, the optical window 250 can effectively expand or contract along with the spacer ring 230, thereby minimizing forces applied onto the optical window 250. In some embodiments, the thermal expansion coefficient of the spacer ring 230 can be within about 3.0×10⁻⁶ inch per inch/° C., of the coefficient of thermal expansion of the optical window 250. In some embodiments, the thermal expansion coefficient of the spacer ring 230 can be within about 1.5×10⁻⁶ inch per inch/° C., of the coefficient of thermal expansion of the optical window 250.

In some embodiments, it can be desirable for the ferrule 210 to exert a residual compressive force on the spacer ring 230. This can aid in keeping the spacer ring 230 firmly in place inside the aperture 212 of the ferrule 210. In an embodiment, the coefficient of thermal expansion of the spacer ring 230 can be slightly lower than the coefficient of thermal expansion of the ferrule 210, resulting in a residual compressive force on the spacer ring 230.

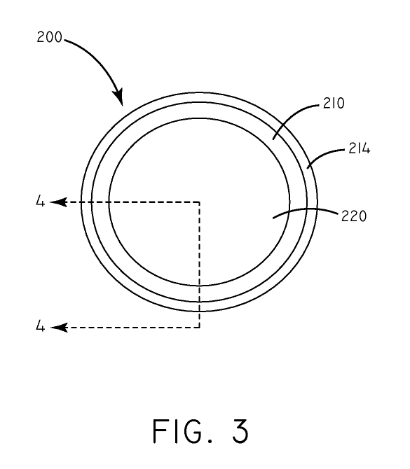

FIG. 3 is a top schematic view of an optical window assembly 200 in accordance with at least one embodiment. The ferrule 210 can define a flange 214, visible from the top of the assembly 200. Additionally, the porous cover 220 can be at least partially disposed within the aperture defined by the ferrule 210.

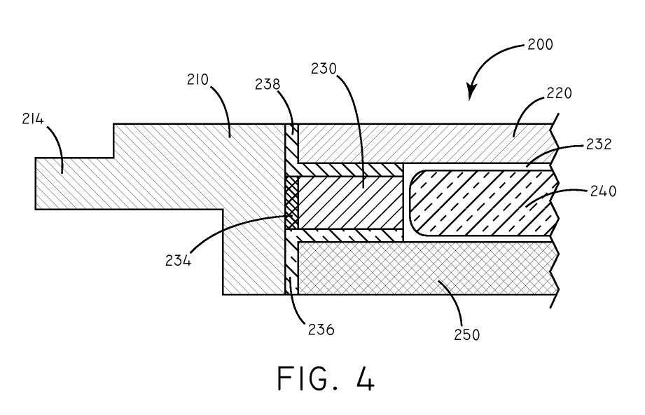

FIG. 4 is a cross sectional view of an optical window assembly 200 as taken along line 4-4 of FIG. 3. The spacer ring 230 is coupled to the ferrule 210 via a first sealing joint 234. In various embodiments, vacuum furnace brazing can be used as a technique to couple the spacer ring 230 to the ferrule 210, though other techniques can also be used. As such, in some embodiments, the first sealing joint 234 can include a brazing material. Many different types of brazing materials can be used. In some embodiments, an active brazing alloy can be used. In at least one embodiment, a gold-based brazing alloy is used. In other embodiments, the first sealing joint 234 can include a weld. Other types of bonding materials could also be used. However, while not intending to be bound by theory, it is believed that a brazing material can be advantageous because of the strength that it lends to the first sealing joint 234. The first sealing joint 234 can form a hermetic seal between the spacer ring 230 and the ferrule 210.

The optical window 250 can be coupled to the ferrule 210 and the spacer ring 230 via a second sealing joint 236. The second sealing joint 236 can form a hermetic seal between the optical window 250, the ferrule 210 and the spacer ring 230. The second sealing joint 236 can be configured so that a any leak path that could be formed is relatively long. In other words, in the event of failure of the second sealing joint 236, contaminant materials would have to pass over a relatively long distance before they could actually enter the interior of the medical device. In some embodiments, the leak path has at least two axes, where one axis is perpendicular to the other. A glass material can be used to form the second sealing joint 236. The glass material can be a bonding glass, solder glass, or sealing glass. As used herein, the term “bonding glass” shall be equivalent to the terms “solder glass” and “sealing glass”. In some embodiments, the glass material can be a glass frit. The glass material of the second sealing joint 236 can have a firing temperature below the softening point of the optical window 250. Techniques used to form the second sealing joint 236 can include the use of air or controlled atmosphere furnaces. In various embodiments the sealing glass would is biocompatible.

In some embodiments, such as the one depicted in FIG. 4, at least one portion of a surface of the optical window 250 can be substantially flush with at least one portion of a surface of the ferrule 210.

The porous cover 220 can be coupled to the ferrule 210 and the spacer ring 230 via a third sealing joint 238. In some embodiments, the third sealing joint 238 can include an adhesive. The adhesive can secure the porous cover 220 in position, thereby sandwiching the chemical sensing element 240 in between the porous cover 220 and the optical window 250 within the optical window assembly 200. In some embodiments, the adhesive can be a silicone rubber medical adhesive such as a polydimethylsiloxane containing composition. Other techniques and compositions can also be used to secure the porous cover 220 in position. However, while not intending to be bound by theory, the use of adhesives can be advantageous because they can form a seal at relatively low temperatures, thereby preventing damage to the chemical sensing element 240 when it is sandwiched into position. In some embodiments, such as the one depicted in FIG. 4, at least one portion of a surface of the porous cover 220 can be substantially flush with at least one portion of a surface of the ferrule 210.

The chemical sensing element 240 can be disposed within the cavity 232 defined by the spacer ring 230. In some embodiments, the chemical sensing element 240 can be bonded to one or more of the porous cover 220, the spacer ring 230, and the optical window 250. However, in other embodiments, the chemical sensing element can simply be loose within the cavity 232.

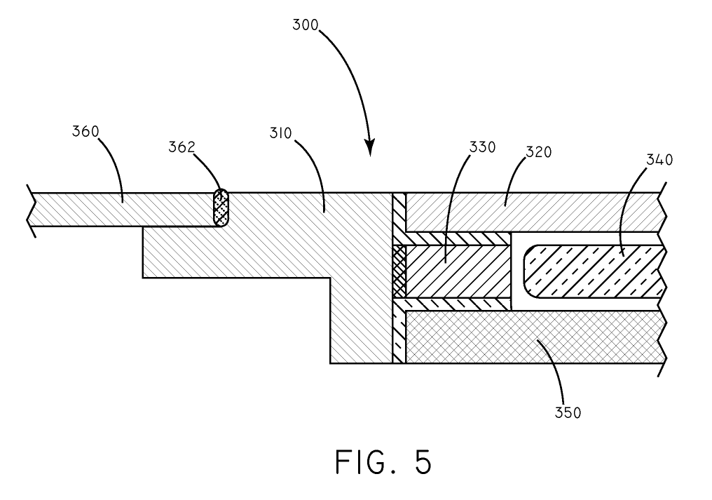

FIG. 5 is a cross sectional schematic view of an optical window assembly 300 in accordance with at least one embodiment. The window assembly 300 in this embodiment can include a ferrule 310, a spacer ring 330, a porous cover 320, a chemical sensing element 340, and an optical window 350. In this view, the window assembly 300 is coupled to a housing wall 360. By way of example, the window assembly 300 can be coupled to the housing wall 360 through a device joint 362. The device joint 362 can form a hermetic seal between the window assembly 300 and the housing wall 360. The device joint 362 can be formed though techniques such as welding or brazing. By way of example, laser welding can be used to couple the window assembly 300 to the housing wall 360. The housing wall 360 can be an element of an implantable medical device such as a cardiac rhythm management device or an implantable monitoring device.

In the embodiment shown in FIG. 5, the optical window 350 faces the interior of the housing 360, and the porous cover 320 faces the outside of the housing 360. This configuration allows for the passage of analytes surrounding the housing 360 in to the chemical sensing element 340. The chemical sensing element can exhibit an optical response which can be detected by other equipment such as an optical detection assembly (not shown).

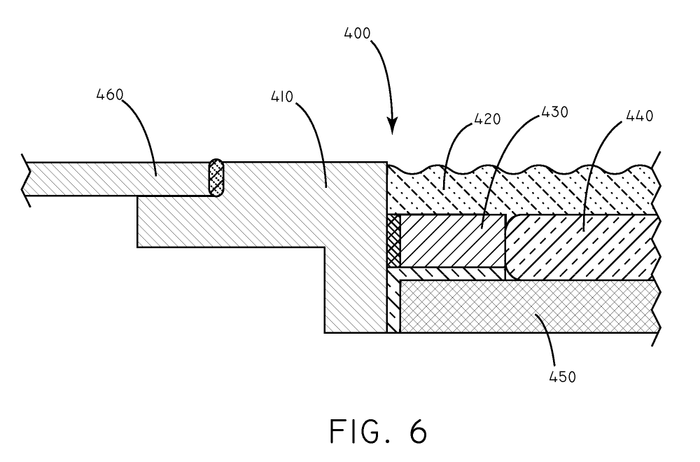

In some embodiments, the optical window assembly can include an encapsulant material. Referring now to FIG. 6, a cross sectional schematic view is shown of an optical window assembly 400 in accordance with at least one embodiment. The window assembly 400 in this embodiment can include a ferrule 410, a spacer ring 430, a chemical sensing element 440, and an optical window 450. The window assembly 400 is coupled to a housing wall 460. In this embodiment the chemical sensing element 440 can be held in place with an encapsulant material 420. The encapsulant material 420 can allow passage of one or more analytes to the chemical sensing element 440. The encapsulant material 420 can include various materials. By way of example, in some embodiments, the encapsulant material 420 can include a porous polymeric matrix.

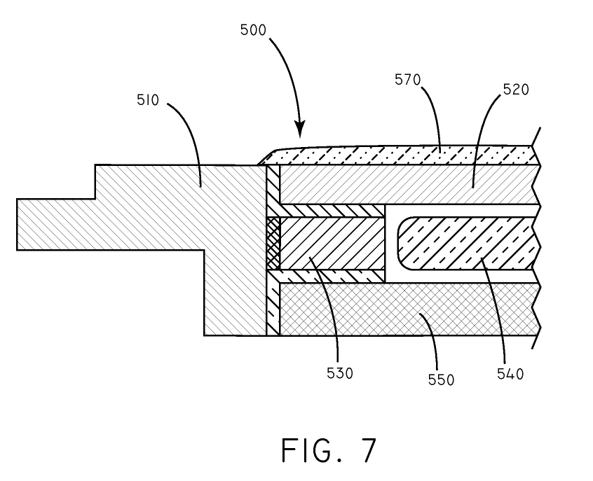

In some embodiments, a layer of material can be disposed on the outside of the porous cover. FIG. 7 is a cross sectional schematic view of an optical window assembly 500 for implantable sensors in accordance with at least one embodiment. The window assembly 500 in this embodiment can include a ferrule 510, a spacer ring 530, a porous cover 520, a chemical sensing element 540, and an optical window 550. In this embodiment the porous cover 520 is covered with a coating 570 (not to scale) such as, for example, a polytetrafluoroethylene (PTFE) layer. The coating 570 can allow passage of one or more analytes. The coating 570 can also serve to modulate the growth habits of cells contacting the coating 570 as well as modulate the immune response of the tissue contacting the coating 570. In some embodiments, the coating 570 can include a layer of hydroxyapatite.

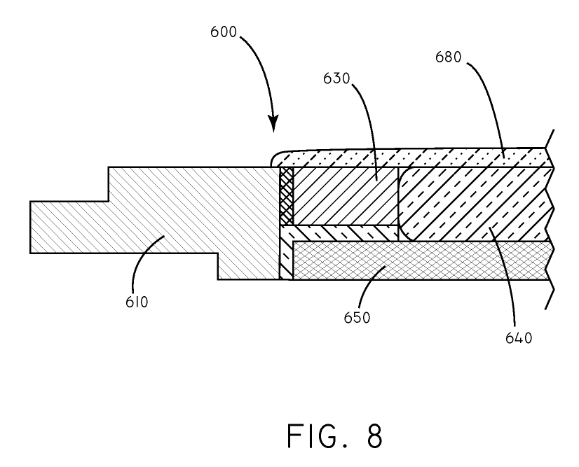

In some embodiments, a porous layer on the outside of the window assembly can be used to retain the chemical sensing element in position. FIG. 8 is a cross sectional schematic view of an optical window assembly 600 in accordance with at least one embodiment. The window assembly 600 in this embodiment can include a ferrule 610, a spacer ring 630, a chemical sensing element 640, and an optical window 650. In this embodiment a porous layer 680 is not disposed within the aperture defined by the ferrule 610, but rather is disposed over the aperture defined by the ferrule 610, substantially covering the spacer ring 630 and the chemical sensing element 640. In some embodiments, the porous layer 680 can substantially cover the chemical sensing element 640 and can be coupled to the spacer ring 630 and/or the ferrule 610. The porous layer 680 can be coupled to the spacer ring 630 and/or the ferrule 610 through a variety of techniques, such as with an adhesive.

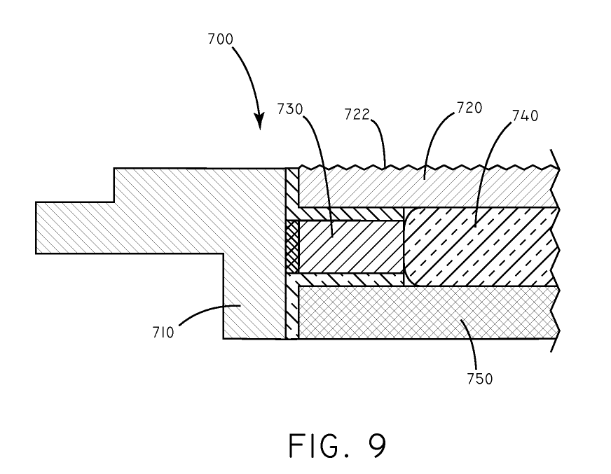

In some embodiments, the outside surface of the porous cover can include topological surface features configured to modulate the growth habits of cells that may interact with the porous cover in vivo. Referring now to FIG. 9, a cross sectional schematic view of an optical window assembly is shown in accordance with at least one embodiment. The window assembly 700 in this embodiment can include a ferrule 710, a spacer ring 730, a porous cover 720, a chemical sensing element 740, and an optical window 750. The outer surface 722 of the porous cover 720 can include topological surface features. These surface features can be configured to modulate the growth habits of cells. In some embodiments, the surface features can include a plurality of peaks and valleys.

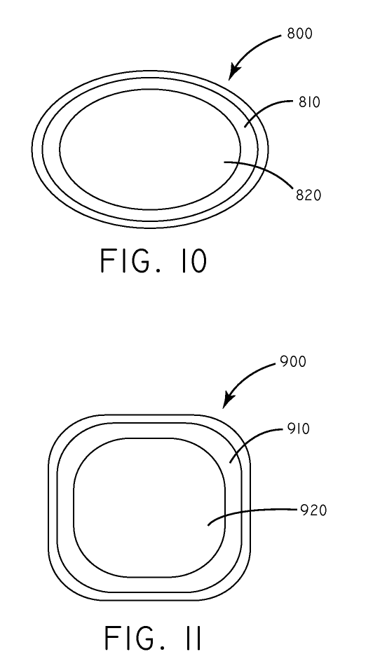

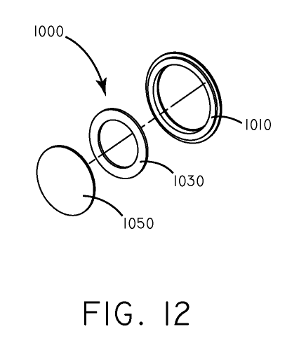

Although the optical window assembly shown in FIGS. 2-4 has a substantially circular shape, it will be appreciated that embodiments herein can include a variety of different shapes. Referring now to FIG. 10, a top schematic view of an optical window assembly 800 in accordance with at least one embodiment is shown. In this embodiment, the ferrule 810 and the porous cover 820 have substantially elliptical shapes. Referring now to FIG. 11, a top schematic view of an optical window assembly 900 having yet a different shape is shown. In this embodiment the ferrule 910 and the porous cover 920 have a rounded rectangular shape. Many other shapes for the optical window assembly are contemplated herein. It will be appreciated that some of the elements, such as the chemical sensing element and/or the porous cover, may be omitted in some embodiments. For example, referring now to FIG. 12, an exploded view of an optical window assembly 1000 is shown in accordance with at least one embodiment wherein the chemical sensing element and the porous cover are omitted. In this embodiment, the optical window assembly 1000 includes a ferrule 1010, a spacer ring 1030, and an optical window 1050.

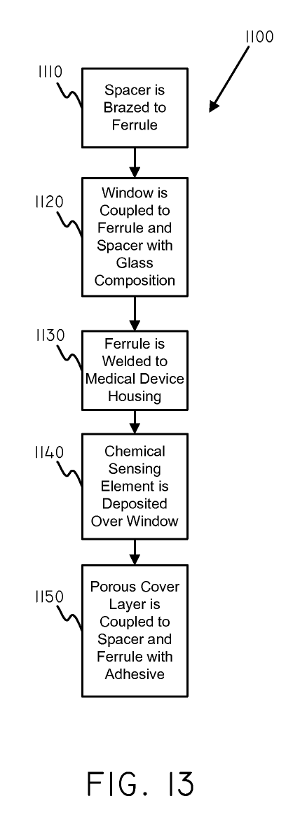

FIG. 13 is a flow diagram of an exemplary method 1100 of manufacturing a medical device in accordance with at least one embodiment. In one operation, the spacer ring is brazed to the ferrule 1110, such as with an active metal braze alloy. Vacuum furnace brazing is one technique that can be used, but other types of brazing are also within the scope of embodiments disclosed herein.

In another operation, the optical window is coupled to the ferrule and the spacer with a glass composition 1120. The optical window can be sealed to the surrounding ferrule using sealing glass, forming a hermetic seal. The sealing glass can have a firing temperature below the softening point of the optical window. In general, this operation can be performed at a lower temperature than the operation of brazing the spacer ring to the ferrule.

In yet another operation, the ferrule is welded to a medical device housing 1130. By way of example, the ferrule can be welded to a medical device housing a laser welding technique. In some embodiments, this operation can be performed after the optical window is coupled to the ferrule and the spacer with a glass composition. In other embodiments, this operation can be performed last, such as after the window assembly is fully assembled.

In another operation, the chemical sensing element can be deposited over the optical window 1140. By way of example, the chemical sensing element can simply be placed on the optical window. As another example, the chemical sensing element can be adhered or fastened to the optical window.

In still another operation, the porous cover is coupled to the spacer ring and the ferrule with an adhesive 1150. In some embodiments, the adhesive can be a silicone rubber medical adhesive such as a polydimethylsiloxane (PDMS) containing composition. It will be appreciated that other adhesives can also be used. By way of example, an epoxy adhesive can be used. The adhesive can be applied at a relatively low temperature, such as ambient room temperature, thereby preventing damage to other elements of the assembly that are already in place.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. An optical window assembly for a medical device, the assembly comprising:
a metal ferrule defining an aperture;
a spacer ring disposed within the aperture, the spacer ring coupled to the metal ferrule with a brazing material; and
an optical window coupled to the metal ferrule and the spacer ring with a bonding glass material; the optical window having a coefficient of thermal expansion within approximately 3.0×10⁻⁶ inch per inch/° C. of the coefficient of thermal expansion of the spacer ring.

2. The optical window assembly of claim 1, the metal ferrule comprising titanium.

3. The optical window assembly of claim 1, the spacer ring comprising a ceramic.

4. The optical window assembly of claim 3, the ceramic of the spacer ring selected from the group consisting of zirconia, alumina, carbides, glass ceramics, and aluminum silicates.

5. The optical window assembly of claim 1, the brazing material comprising an active brazing alloy.

6. The optical window assembly of claim 1, wherein the coefficient of thermal expansion of the spacer ring is lower than the coefficient of the metal ferrule, the metal ferrule exerting a residual compressive force on the spacer ring.

7. The optical window assembly of claim 1, further comprising a porous cover coupled to the spacer ring and the metal ferrule, the porous cover comprising a biocompatible material.

8. The optical window assembly of claim 7, the porous cover comprising a material selected from the group consisting of ceramics, metals, and polymers.

9. The optical window assembly of claim 8, the porous cover comprising a material selected from the group consisting of porous alumina, porous platinum, porous titanium, platinum plated porous titanium, sintered titanium, sintered iridium, and sintered gold.

10. The optical window assembly of claim 7, the porous cover comprising topographic surface features configured to modulate cellular responses.

11. The optical window assembly of claim 1, further comprising a chemical sensing element disposed between the porous cover and the optical window.

12. The optical window assembly of claim 1, the bonding glass material comprising a glass frit.

13. The optical window assembly of claim 1, the optical window comprising a material selected from the group consisting of sapphire (aluminum oxide), soda lime glass, and borosilicate glass.

14. The optical window assembly of claim 1, the optical window having a coefficient of thermal expansion within approximately 1.5×10⁻⁶ inch per inch/° C. of the coefficient of thermal expansion of the spacer ring.

15. An implantable medical device comprising:
a hermetically sealed housing defining an interior volume; and
an optical window assembly coupled to the housing, the assembly comprising
a metal ferrule defining an aperture;
a spacer ring disposed within the aperture, the spacer ring coupled to the metal ferrule with a brazing material; and
an optical window coupled to the metal ferrule and the spacer ring with a bonding glass material; the optical window having a coefficient of thermal expansion within approximately 3.0×10⁻⁶ inch per inch/° C. of the coefficient of thermal expansion of the spacer ring.

16. The implantable medical device of claim 15, wherein the coefficient of thermal expansion of the spacer ring is lower than the coefficient of the metal ferrule, the metal ferrule exerting a residual compressive force on the spacer ring.

17. The implantable medical device of claim 15, further comprising a porous cover coupled to the spacer ring and the metal ferrule, the porous cover comprising a biocompatible material.

18. The implantable medical device of claim 15, further comprising a chemical sensing element disposed between the porous cover and the optical window.

19. A method of manufacturing a medical device, the method comprising:
brazing a spacer ring to a metal ferrule;
coupling an optical window to the spacer ring and the metal ferrule with a bonding glass material;
depositing a chemical sensing element over the optical window; and
coupling a porous cover layer to the spacer ring and the ferrule with an adhesive.

20. The method of claim 19, further comprising the step of welding the metal ferrule to a medical device housing.
